(19) United States
(12) Patent Application Publication
(10) Pub. No.: US 2026/0027022 A1
(43) Pub. Date: Jan. 29, 2026

(54) ENCAPSULATION COMPOSITIONS COMPRISING LIQUID CRYSTAL PARTICLES AND COSMETIC OR PHARMACEUTICAL COMPOSITIONS AND METHODS FOR PRODUCING THE SAME

(71) Applicant: ELC Management LLC, MELVILLE, NY (US)

(72) Inventors: Weining ZHOU, Shanghai (CN); Kefeng TONG, Shanghai (CN); Lingling LUO, Shanghai (CN); Gang LIN, Shanghai (CN); Jing CHENG, Shanghai (CN); Francis Martin FRIEL, Shanghai (CN)

(21) Appl. No.: 19/101,575

(22) PCT Filed: Aug. 9, 2022
(86) PCT No.: PCT/CN2022/111085

§ 371 Date: Feb. 5, 2025

(51) Int. Cl. A61K 8/11

(52) U.S. Cl. CPC A61K 8/11; A61K 2800/10; A61K 2800/56; A61K 2800/596; A61K 2800/652; A61K 2800/80

(57) ABSTRACT

The present application provides an encapsulation composition comprising liquid crystal particles, wherein each of the liquid crystal particles comprises a core and a liquid crystal shell, wherein the core contains an active ingredient and the liquid crystal shell contains a fatty alcohol, a first surfactant, and an optional lipid. The present application further provides a cosmetic or pharmaceutical composition comprising the encapsulation composition. The method for preparing the encapsulation composition is also provided.

### FIELD OF THE DISCLOSURE

The present disclosure generally relates to encapsulation of active ingredients, and more particularly to liquid crystal encapsulation systems and methods. More particularly, the present disclosure relates to encapsulation compositions comprising liquid crystal particles, cosmetic or pharmaceutical compositions comprising the encapsulation composition, and methods for producing the same.

### BACKGROUND

Cosmetics generally include a complex mixture of various ingredients including, but not limited to, water, oils, surfactants, pigments, preservatives and the like employed in different cosmetic compositions including, but not limited to lotions, creams, emulsions, gels, sprays and the like, with different efficacies including, but not limited to, whitening, anti-wrinkling, and sunscreens. Active ingredients may be introduced to support the above-mentioned efficacies.

Some active ingredients may limit development of cosmetic formulations, such as limiting the delivery of a required texture or feeling when applied to a user's skin. For example, diethylamino hydroxybenzoyl hexyl benzoate and ethylhexyl triazone are ultraviolet (UV) filters widely used in sunscreen products, especially those having high sun protection factor (SPF). However, these types of UV filters may result in a greasier feeling on a user's skin, especially as higher loads of the filters are included in sunscreen products. An oil is generally added as a solvent to dissolve these UV filters, resulting in a greasy feeling upon application to skin and limiting freedom of cosmetic formulation.

Some active ingredients such as retinol or its derivatives may have potential side effects upon application such as redness, irritation, skin flaking, sun-sensitive etc. Furthermore, retinol is sensitive towards oxidation and has poor aqueous solubility.

Pharmaceutical compositions generally include a complex mixture of various ingredients. It is also desirable to encapsulate pharmaceutical active ingredients to provide improved properties.

An encapsulation system can be provided to encapsulate active ingredients to reduce or eliminate side effects, to improve the texture or feeling when applied to skin, or to preserve the advantageous properties of the active ingredients during storage. Although various encapsulation systems are known in the art, such as solid lipid nanoparticles (SLN), nanostructured lipid carrier (NLC) and liquid crystal encapsulation, it is desirable to provide a liquid crystal encapsulation system with improvements over prior art, such as longer durability and stability, lower burst release and leakage of cored actives, higher compatibility with formula systems, higher encapsulated capability, and/or more simple or cost-effective producing process.

The present application provides at least one of above improvements.

### SUMMARY

In an aspect, the present application provides an encapsulation composition comprising liquid crystal particles, wherein each of the liquid crystal particles comprises a core and a liquid crystal shell, wherein the core contains an active ingredient and the liquid crystal shell contains a fatty alcohol, a first surfactant, and an optional lipid.

According to an embodiment, the first surfactant can be a gemini surfactant, preferably a gemini amphiphilic surfactant; and more preferably the first surfactant can be sodium dilauramidoglutamide lysine (DLGL). The first surfactant can range from 0.1-20%, preferably 0.1-10%, more preferably 0.1-5% by weight of the encapsulation composition. According to an embodiment, the fatty alcohol can be selected from the group consisting of myristyl alcohol, cetyl alcohol, cetyl/stearyl alcohol, stearyl alcohol, arachyl alcohol, behenyl alcohol, and mixtures thereof. The fatty alcohol can range from 0.1-50%, preferably 1-20%, more preferably 1-10% by weight of the encapsulation composition. According to an embodiment, the active ingredient generally is lipophilic. The active ingredient can range from 1-70%, preferably 5-65% by weight, more preferably 5-60% by weight of the encapsulation composition. Preferably, the active ingredient is selected from the group consisting of lipophilic ultraviolet (UV) filters, retinol and its derivatives, and mixtures thereof.

According to an embodiment, the active ingredient can be a lipophilic ultraviolet (UV) filter and the lipophilic ultraviolet (UV) filter can range from 1-70%, preferably 5-60% by weight of the encapsulation composition. Preferably the lipophilic ultraviolet (UV) filter is selected from the group consisting of ethylhexyl triazone, diethylamino hydroxybenzoyl hexyl benzoate, bis-ethylhexyloxyphenol methoxyphenyl triazine and mixtures thereof.

According to an embodiment, the active ingredient can be retinol and/or its derivatives and the retinol and/or its derivatives can range from 1-70%, preferably 5-60% by weight of the encapsulation composition.

According to an embodiment, the first surfactant can range from 0.1-20%, preferably 0.1-10%, more preferably 0.1-5% by weight of the encapsulation composition; the fatty alcohol can range from 0.1-50%, preferably 1-20%, more preferably 1-10% by weight of the encapsulation composition; and/or the active ingredient can range from 1-70%, preferably 5-60% by weight of the encapsulation composition.

According to an embodiment, the encapsulation composition can be formed by mixing a first mixture comprising a polyol, the first surfactant and water and a second mixture comprising the active ingredient, the fatty alcohol and the optional lipid. The liquid crystal particles are formed during the mixing.

The polyol can be selected from polyols having two or more hydroxyls and 3-12, preferably 3-6 carbon atoms in the molecule. Preferably, the polyol is selected from the group consisting of glycerin, 1,2-propanediol, 1,3-propanediol, 1,4-butanediol, 1,3-butylene glycol, dipropylene glycol, 1,2-pentandiol, 2-methyl-pentan-2,4-diol, 1,2-hexanediol, sorbitol, 1,2-octanediol, and mixtures thereof. More preferably the polyol is glycerin, sorbitol and mixtures thereof. The polyol can range from 10-80%, preferably 20-60% by weight of the encapsulation composition.

Water can range from 1-10% by weight of the encapsulation composition.

According to an embodiment, the lipid can be selected from the group consisting of triglycerides, hydrocarbons, ester, fatty acids, waxes, sphingolipids, phospholipids, cholesterols, and mixtures thereof. The lipid can range from 0-70% by weight of the encapsulation composition.

According to an embodiment, the ratio by weight of the second mixture to the first mixture can range from 0.1-10, preferably 0.2-5.

The second mixture can be in the form of a solid, a semi-solid, a paste, or a liquid paste at 25 degrees Celsius.

According to an embodiment, the polyol can comprise glycerin; the first surfactant can comprise sodium dilauramidoglutamide lysine; the active ingredient can comprise diethylaminohydroxybenzoyl hexyl benzoate and/or ethylhexyl triazone; and the fatty alcohol can comprise behenyl alcohol.

According to an embodiment, the liquid crystal shell can contain the lipid and the active ingredient can comprise retinol and/or its derivatives.

In another aspect, the present application provides a cosmetic or pharmaceutical composition comprising the encapsulation composition of present application.

According to an embodiment, the cosmetic or pharmaceutical composition can further comprise a second surfactant to control the particle size of the liquid crystal particles. Preferably the particle size of the liquid crystal particles is in a range from 10 nm-300 μm, preferably 50 nm-100 μm, more preferably 200 nm-100 μm in the presence of the second surfactant. The second surfactant can be selected from the group consisting of fatty acid esters of glycerol, fatty acid esters of sorbitol, fatty acid ethoxylates, and mixtures thereof. As an example, the second surfactant can be polyglyceryl-3 methylglucose distearate.

The second surfactant can range from 0.5-10%, preferably 1-10%, more preferably 1-5% by weight of total of the encapsulation composition, the at least one second surfactant and any other ingredients in the cosmetic or pharmaceutical composition.

In a further aspect, the present application provides a method for preparing the encapsulation composition of present application, including:
a) providing a first mixture comprising a polyol, a first surfactant, and water and a second mixture comprising an active ingredient, a fatty alcohol and an optional lipid;
b) optionally, mixing the first mixture and/or mixing the second mixture;
c) mixing the first mixture and the second mixture at a temperature equal to or higher than the melting point of the fatty alcohol; and
d) cooling the resulting mixture of step c) to room temperature to provide the encapsulation composition.

In an embodiment, the mixing of the first mixture is conducted at a temperature equal to or higher than the melting point of the fatty alcohol and the mixing of the second mixture is conducted at a temperature equal to or higher than the melting point of the fatty alcohol.

In an embodiment, mixing the first mixture and the second mixture includes adding the second mixture into the first mixture.

Liquid crystal (LC) encapsulation of present application can reduce or eliminate the amount of solvent needed to dissolve an active ingredient such as an UV filter, by encapsulating the UV filter in a core. Thus a solvent-free liquid crystal can be provided, as no solvent is needed to dissolve the active ingredients. Accordingly, more concentrated active ingredients can be encapsulated in a core. Accordingly, a high SPF value may be delivered with a desired sensory feeling on the skin.

Retinol and its derivatives may have potential side effects upon application such as redness, irritation, skin flaking, sun-sensitive etc. Furthermore, retinol is sensitive towards oxidation and has poor aqueous solubility. The liquid crystal encapsulation of present application can reduce or eliminate such undesirable effects.

The liquid crystal encapsulation systems and methods of present application are feasible for use at high temperatures. The liquid crystal particles of present application are more durable over time. A high liquid crystal particle size controllability can be achieved easily in present application through addition of a second surfactant and a proper mixing.

### BRIEF DESCRIPTION OF THE DRAWINGS

For a more complete understanding of this disclosure, reference is now made to the following description, taken in conjunction with the accompanying drawing, in which:

FIG. 1 is a schematic view of the liquid crystal particle according to the present application;

FIG. 2 schematically depicts the liquid crystal particles including core actives after application to the skin according to the present application.

FIG. 3 is a micrograph of the cosmetic composition of Example 1 before stability test.

FIG. 4 is a micrograph of the comparative cosmetic composition of Example 2 before stability test.

FIG. 5 is a micrograph of the cosmetic composition of Example 1 after stability test (freeze-thaw test).

FIG. 6 is a micrograph of the cosmetic composition of Example 1 after stability test (50° C.).

### DETAILED DESCRIPTION

The present application relates to encapsulation of an active ingredient by a liquid crystal shell. The active ingredient encapsulated by the liquid crystal shell can be present in the form of particles. The present application thus provides an encapsulation composition comprising liquid crystal particles, wherein each of the liquid crystal particles comprises a core and a liquid crystal shell encapsulating the core, wherein the core contains an active ingredient and the liquid crystal shell contains a fatty alcohol, a first surfactant, and an optional lipid. The liquid crystal particle according to present application thus includes the core encapsulated by the liquid crystal shell.

The term “optional” means the lipid can be either present or not present in the shell. When lipid is not present in the shell, it means lipid is not used in the preparation of the encapsulation composition.

According to an embodiment, the core contains an active ingredient and an optional lipid and the liquid crystal shell contains a fatty alcohol, a first surfactant, and an optional lipid. The lipid in the core and the lipid in the liquid crystal shell are the same.

The liquid crystal particles formed in present invention can be characterized by Maltese cross. The appearance of Maltese cross under polarizing microscope is a typical feature of the formation of liquid crystal structure.

“Active ingredient” means compounds, substances, molecules, and mixtures that are used in cosmetics in order to bring about a favorable effect, or means compounds, substances, molecules, and mixtures that has a therapeutic effect. For cosmetics, it may be desirable in some cases that the active ingredient is transported into the epidermal layer in order to interact with or protect the epidermis and deeper skin layers.

The active ingredient encapsulated by the liquid crystal shell can be any lipophilic active ingredients. Preferably the active ingredient is selected from those having low compatibility with a cosmetic composition or those having potential side effects upon application such as redness, irritation, skin flaking etc and need to be encapsulated before introduced into a cosmetic composition. Preferably, the active ingredient is selected from the group consisting of lipophilic ultraviolet (UV) filters, retinol and its derivatives, and mixtures thereof. The active ingredient can range from 1-70%, preferably 5-60% by weight of the encapsulation composition.

The first surfactant can be a gemini surfactant, preferably a gemini amphiphilic surfactant. “Gemini surfactant” generally is known in the art. Gemini surfactants can have dimeric structures, composed of two hydrophobic chains and two hydrophilic heads, linked by a spacer at or near the head groups. According to a preferred embodiment, the first surfactant can be sodium dilauramidoglutamide lysine (DLGL).

The first surfactant can range from 0.1-20%, preferably 0.1-10%, more preferably 0.1-5% by weight of the encapsulation composition.

Any fatty alcohol can be used in present application provided the formation of liquid crystal structure as described herein is not negatively affected. According to an embodiment, the fatty alcohol can be selected from the group consisting of myristyl alcohol, cetyl alcohol, cetyl/stearyl alcohol, stearyl alcohol, arachyl alcohol and behenyl alcohol, and any combination thereof.

According to an embodiment, the at least one fatty alcohol can be selected depending on the desired texture/sensory feel of cosmetic compositions. For example, inclusion of behenyl alcohol may provide a stronger texture for cosmetic compositions while cetyl alcohol may provide a lower viscosity for cosmetic compositions.

The fatty alcohol can range from 0.1-50%, preferably 1-20%, more preferably 1-10% by weight of the encapsulation composition.

Lipids are well known in the art and lipids commonly used in cosmetics can be used in present application. According to an embodiment, the lipids used in present application can be selected from the group consisting of triglycerides, hydrocarbons, ester, fatty acids, waxes, sphingolipids, phospholipids, cholesterols and mixtures thereof. In present application, the lipid can range from 0-70% by weight of the encapsulation composition.

The present application also provides a method for preparing the encapsulation composition as described above. The method can include a) providing a first mixture comprising a polyol, a first surfactant, and water, and a second mixture comprising an active ingredient, a fatty alcohol and an optional lipid; b) optionally, mixing the first mixture and/or mixing the second mixture; c) mixing the first mixture and the second mixture together at a temperature equal to or higher than the melting point of the fatty alcohol; and d) cooling the resulting mixture of step c) to room temperature to provide the encapsulation composition. The term “optionally” means the step b) is either present or not present in the method. When step b) is present, the method includes steps a), b), c) and d), and when step b) is not present, the method includes steps a), c) and d).

“Room temperature” in present application means a temperature of about 25° C.

Water used in the first mixture can be any water that can be used in cosmetic or pharmaceutical compositions, including, but not limited to, deionized water, purified water and the like. Water can range from 1-10% by weight of the encapsulation composition.

The first surfactant, the fatty alcohol and the active ingredient used in the method are the same as those described in said encapsulation composition.

The polyol of present application can be any polyol having two or more hydroxyls and 3-12, preferably 3-6 carbon atoms in the molecule. In an embodiment, the polyol used in the method can be selected from the group consisting of glycerin, 1,2-propanediol (propyleneglycol), 1,3-propanediol (for example ZEMEA™ marketed by Duponte Tate & Lyle), 1,4-butanediol, 1,3-butylene glycol, dipropylene glycol, 1,2-pentandiol, 2-methyl-pentan-2,4-diol (for example Diolane), 1,2-hexanediol, sorbitol, 1,2-octanediol, dipropylene glycol propyl ether (for example Dowanol® DPnP marketed by Dow Chemicals), and dipropylene glycol isobornylether (for example Pribelance™ Clear marketed by CARB) and mixtures thereof. Preferably the polyol is glycerin, sorbitol and mixtures thereof. The polyol can range from 10-80%, preferably 20-60% by weight of the encapsulation composition (i.e. by the weight of the total the first mixture and the second mixture).

Heat may be applied to mix the first mixture and/or the second mixture, and the heating temperature should be at least equal to or higher than the melting point of the fatty alcohol used in the second mixture. In a preferred embodiment, the mixing of the first mixture is conducted at a temperature equal to or higher than the melting point of the fatty alcohol and the mixing of the second mixture is conducted at a temperature equal to or higher than the melting point of the fatty alcohol. When a mixture of fatty alcohols is used, the heating temperature should be equal to or higher than the melting point of the fatty alcohol having the highest melting point in the mixture.

The heat can be provided by any heating means generally known in the art. There are no particularly limitations to the means of heating.

In a preferred embodiment, mixing the first mixture and the second mixture together includes adding the second mixture into the first mixture, preferably under heating and mixing, wherein the temperature is increased to equal to or higher than the melting point of the fatty alcohol.

Any appropriate mixing means known in the field can be used in the method, for example mixing with stirring. Any proper mixing method may be utilized as permitted in cosmetic or pharmaceutical production. Those skilled in the art can select and adjust the mixing means, for example according to the particle size of the liquid crystal particles to be obtained in the encapsulation composition.

In an embodiment, the resulting mixture of step c) can be cooled naturally in step d).

According to an embodiment, the first surfactant can range from 0.1-20%, preferably 0.1-10%, more preferably 0.1-5% by weight of the encapsulation composition; the fatty alcohol can range from 0.1-50%, preferably 1-20%, more preferably 1-10% by weight of the encapsulation composition; the active ingredient(s) can range from 1-70%, preferably 5-60% by weight of the encapsulation composition; the polyol can range from 10-80%, preferably 20-60% by weight of the encapsulation composition; and water can range from 1-10% by weight of the encapsulation composition.

Those skilled in the art can easily select the ratio between the first mixture and the second mixture. For example, the ratio can be adjusted according to the level of active ingredients to be incorporated into the encapsulation composition. In an embodiment, the ratio by weight of the second mixture to the first mixture can range from 0.1-10, preferably 0.2-5.

It should be appreciated that the weight percentages of ingredients are based on the weight of the encapsulation composition, i.e. based on the combination of the first mixture and the second mixture.

It should be appreciated that the second mixture may take various forms including, but not limited to, a solid, a semi-solid, a paste or a liquid paste under 25 degrees Celsius. The form may mainly depend on the selected fatty alcohol and lipid (if present).

It is surprisingly found in present application that the liquid crystal particles according to present application are more stable than liquid crystal particles known in the art.

The method of present application is simple and thus has low requirements for equipment. The method of present application can reduce energy consumption because high-energy consumption mixing procedures such as ultrasonic and high-pressure homogenization are not essential.

The present application provides a liquid crystal encapsulation technology that can reduce or eliminate the amount of solvent needed to dissolve one or more lipophilic active ingredients by encapsulating the active ingredient(s) in a core.

According to embodiment, the active ingredients in the core can include a UV filter, preferably a lipophilic UV filter. The lipophilic UV filters can be those commonly used in the field of cosmetics. As examples, the lipophilic UV filters can be ethylhexyl triazone (for example UVINUL T150), diethylamino hydroxybenzoyl hexyl benzoate (for example UVINUL A PLUS GRANULAR), and bis-ethylhexyloxyphenol methoxyphenyl triazine (for example TINOSORB S). According to present application, a high SPF value can be delivered with a desired sensory feeling when applied to the skin, using the lipophilic UV filter as the active ingredient. The lipophilic UV filter can range from 1-70%, preferably 5-65% by weight, more preferably 5-60% by weight of the encapsulation composition. According to an embodiment, the lipophilic UV filter can range from 20-70%, preferably 30-65% by weight, more preferably 40-65% by weight of the encapsulation composition.

According to an embodiment, the active ingredient in the core can include retinol and/or its derivatives. Retinol is a synthetic derivative of vitamin A, a fat-soluble vitamin common in carrots, eggs and potatoes, which is a very popular skin care ingredient for aiding lots of skin concerns, such as fine lines, pigmentation and acne. But retinol has several potential side effects upon application such as redness, irritation, skin flaking, sun-sensitive etc. Furthermore, retinol is sensitive towards oxidation and has poor aqueous solubility. The encapsulation composition of present application comprising liquid crystal particles can encapsulate retinol in the core so as to preserve the retinol properties during storage, enhance its physiological potencies and eliminate the side effects of retinol. The retinol and its derivatives can range from 1-70%, preferably 5-60% by weight of the encapsulation composition.

According to an embodiment, the liquid crystal shell in the liquid crystal particle contains the lipid when the core contains retinol or its derivatives.

The liquid crystal encapsulation technology of present application has a wider applicability compared with conventional liquid crystal encapsulation technology. For example, the liquid crystal encapsulation technology of present application is feasible for use at high temperatures (up to 80-90 degrees Celsius depending on the melting point of active ingredients to be encapsulated). Accordingly, active ingredients having high melting points can be more easily introduced into cosmetic compositions. Moreover, the liquid crystal particles are more durable over time compared with liquid crystal particles prepared by conventional liquid crystal encapsulation technology.

The encapsulation composition according to present application can include solvent-free liquid crystal particles, as no solvent is needed to dissolve the active ingredients. Accordingly, the core of the liquid crystal particles can comprise a higher level of active ingredients.

The present application further provides a cosmetic or pharmaceutical composition comprising the encapsulation composition of present application.

In present application, a cosmetic or pharmaceutical composition comprising the encapsulation composition means the encapsulation composition is used in the preparation of the cosmetic or pharmaceutical composition. Those skilled in the art will appreciated that the encapsulation composition will mix with other ingredients in the cosmetic or pharmaceutical composition during the preparation of the cosmetic or pharmaceutical composition, and the liquid crystal particles in the encapsulation particles will mix with other ingredients in the cosmetic or pharmaceutical composition. Generally the liquid crystal particles will be dispersed in the cosmetic or pharmaceutical composition. In present application, “cosmetic composition” means a composition for affecting the appearance of or beautifying the face, skin, hair, nails or other keratinous materials. Cosmetic compositions are well known in the art.

The cosmetic composition of present application can be formulated into various forms, such as lotions, creams, emulsions, gels, sprays and the like.

The level of the encapsulation composition of present application in the cosmetic composition can be easily selected and determined by those skilled in the art, according to the level of the active ingredient to be incorporated into the cosmetic composition.

The cosmetic composition of present application can comprise various ingredients usually used in cosmetic products, which are known in the art. In particular, the ingredients can be selected and combined according to the forms of cosmetic composition and/or according to the desired properties or characteristics of the cosmetic composition. As examples, the ingredients can include, but not limited to, one or more of antioxidants, chelating agents, coloring agents, emollients, emulsifiers, fragrances, humectants, lubricants, preservatives, stabilizers, surfactants, viscosity modifiers, or any combination thereof. The amounts of the ingredients can be easily selected and adjusted by those skilled in the art. For example, the ingredients can be used in the levels generally adopted in cosmetic products.

Alternatively or in addition, the cosmetic composition of present application can comprise at least one additional active ingredient in addition to the active ingredient comprised in the encapsulation composition described above. The additional active ingredients can be ingredients generally known in the cosmetics and can be selected according to the desired effect(s) to be provided by the cosmetic composition. For example, the additional active ingredients can be selected from radical scavengers, ultraviolet absorbers, vitamins, peeling active ingredients, retinoids, anti-acne active ingredients, tanning active ingredients, anti-inflammatory active ingredients, anti-microbial active ingredients, antifungal active ingredients, skin lightening agents, skin energizing agents, skin healing agents, moisturizing active ingredients and mixtures thereof.

The amounts of the additional active ingredients can be easily selected and adjusted by those skilled in the art. For example, the additional active ingredients can be used in levels generally adopted in cosmetic products so as to provide the desired effects.

According to a preferred embodiment, the cosmetic composition can further comprise a second surfactant. The particle size of the liquid crystal particles can be further adjusted or controlled by the second surfactant. Preferably the particle size of the liquid crystal particles is controlled to be in a range from 10 nm-300 μm, preferably 50 nm-100 μm, more preferably 200 nm-100 μm due to the presence of the second surfactant. The second surfactant can be selected from the group consisting of fatty acid esters of glycerol, fatty acid esters of sorbitol, fatty acid ethoxylates, and mixtures thereof. As an example, the second surfactant can be polyglyceryl-3 methylglucose distearate. The second surfactant can range from 0.5-10%, preferably 1-10%, more preferably 1-5% by weight of total of the encapsulation composition, the at least one second surfactant and any other ingredients in the cosmetic composition. According to an embodiment, the second surfactant can be incorporated into the cosmetic composition during the preparation of the cosmetic composition.

The cosmetic composition of present application can be obtained by mixing together the encapsulation composition of the present application, and all other ingredients in the cosmetic composition (including the second surfactant in present). The encapsulation composition of present application can be added into a third mixture under mixing so as to provide the cosmetic composition. The third mixture can comprise all other ingredients in the cosmetic composition other than the encapsulation composition. Any proper mixing method can be utilized as permitted in cosmetic production provided it can provide the cosmetic composition. Those skilled in the art can select and adjust the type of mixing and/or the mixing speed easily. High-energy consumption mixing procedures such as ultrasonic and high-pressure homogenization can be used, but not an essential operation. According to an embodiment, high-energy consumption mixing procedures are not used in the preparing of the cosmetic composition of present application.

According to a preferred embodiment, for the cosmetic composition of present application, the polyol is selected from the group consisting of glycerin, sorbitol, and mixtures thereof; the first surfactant is sodium dilauramidoglutamide lysine; the active ingredient encapsulated is selected from the group consisting of diethylaminohydroxybenzoyl hexyl benzoate, ethylhexyl triazone, and mixtures thereof; the fatty alcohol is behenyl alcohol; and the second surfactant is polyglyceryl-3 methylglucose distearate. In another preferred embodiment, the cosmetic composition of present application comprises glycerin or sorbitol as the polyol, sodium dilauramidoglutamide lysine as the first surfactant, diethylaminohydroxybenzoyl hexyl benzoate and ethylhexyl triazone as the active ingredients encapsulated, behenyl alcohol as the fatty alcohol, polyglyceryl-3 methylglucose distearate as the second surfactant and ethylhexyl methoxycinnamate as an additional UV filter.

In present application, “pharmaceutical composition” means a composition comprising an active ingredient that has a therapeutic effect. Pharmaceutical compositions are well known in the art. The pharmaceutical composition of present application can be formulated into various dosage forms, such as syrups, capsules, tablets, solutions, suspensions, ointments and gels.

The level of the encapsulation composition of present application in the pharmaceutical composition can easily selected and determined by those skilled in the art, according to the level of the active ingredient to be incorporated into the pharmaceutical composition.

The pharmaceutical composition according to present application can also contain pharmaceutical-acceptable ingredients, including but not limited to carriers, excipients, disintegrants, buffering agents, emulsifiers, suspending agents, sedatives, stabilizers, preservatives, antiseptics, physiological saline, and so forth. Those skilled in the art can select such additional ingredients according to the dosage forms of the pharmaceutical compositions.

The pharmaceutical composition according to the present application can be manufactured using methods commonly known in the pertinent technical fields. Those skilled in the art can easily select the proper manufacturing method according to the dosage forms of the pharmaceutical compositions.

It should be appreciated that inclusion of the polyol in present application can promote the formation of liquid crystal structures in the first surfactant (e.g., sodium dilauramidoglutamide lysine) and at least one fatty alcohol (e.g., behenyl alcohol). In addition, active ingredients can be encapsulated as core materials, and the active ingredients are more easily remain on the user's skin when applied.

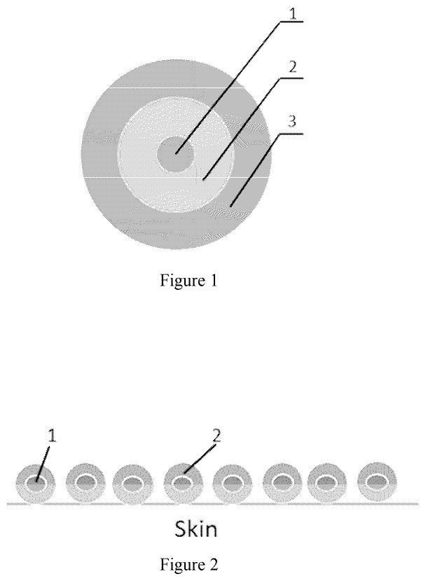

FIG. 1 is a schematic view of the liquid crystal particle according to the present application, depicting the encapsulation of core actives through a liquid crystal structure. As shown in FIG. 1, the core (1) is encapsulated by the liquid crystal shell (2), and the liquid crystal shell (2) is surrounded by a continuous phase (3), such as a continuous phase of a cosmetic composition. FIG. 2 schematically depicts the liquid crystal particles including core actives after application to the skin according to the present application. The continuous phase disappears after the cosmetic composition of present application is applied to the skin. Thus, the liquid crystal particles with active ingredients in the core (1) remain on the skin, wherein core (1) is surrounded by liquid crystal shell (2).

The following Examples are provided to further illustrate the present application.

### EXAMPLES

### Example 1

An encapsulation composition of present application was prepared by propeller mixing ingredients in mixture 1 under 85° C. till uniform and propeller mixing ingredients in mixture 2 under 85° C. till uniform; and adding mixture 2 into mixture 1 under propeller mixing till uniform and cooled down to room temperature. The first mixture and the second mixture are shown in table below.

Example 1 Sunscreen by liquid crystal technology according present application

| Trade Name | INCI | Level % | mixture | remarks |
|---|---|---|---|---|
| GLYCERINE 99.7% USP | GLYCERIN | 5 | 1 | polyol |
| KOSHER RSPO MB | | | | |
| PELLICER L-30 | WATER/SODIUM DILAURAMIDOGLUTAMIDE | 1 | 1 | First surfactant |
| | LYSINE (30% in water) | | | |
| PURIFIED WATER | WATER | 1 | 1 | water |
| UVINUL A PLUS GRANULAR | DIETHYLAMINO HYDROXYBENZOYL HEXYL | 9 | 2 | Actives to be |
| | BENZOATE | | | encapsulated |
| UVINUL T150 | ETHYLHEXYL TRIAZONE | 3 | 2 | |
| BEHENYL ALCOHOL 65 | BEHENYL ALCOHOL | 2 | 2 | Fatty alcohol |
| PURIFIED WATER | WATER | qs to 100% | 3 | water |
| TEGO CARE 450 MB | POLYGLYCERYL-3 METHYLGLUCOSE | 3 | 3 | Second surfactant |
| | DISTEARATE | | | |
| PAROSL MCX | ETHYLHEXYL METHOXYCINNAMATE | 7.5 | 3 | Additional UV filter |

The mixture 3 was mixed under 85° C. till uniform and cooled down to room temperature. The encapsulation composition was added into the mixture 3 by low speed homogenization at 2000-3000 rpm till uniform so as to provide a cosmetic composition (sunscreen emulsion).

### Comparative Example 2

Example 2 Sunscreen by traditional liquid crystal technology

| Trade Name | INCI | Level % | mixture | remarks |
|---|---|---|---|---|
| PURIFIED WATER | WATER | qs to 100% | 1 | water |
| GLYCERINE 99.7% USP | GLYCERIN | 5 | 1 | polyol |
| KOSHER RSPO MB | | | | |
| CARBOPOL 980 | CARBOMER | 0.02 | 1 | thickener |
| UVINUL A PLUS | DIETHYLAMINO HYDROXYBENZOYL HEXYL | 9 | 2 | Same actives as in |
| GRANULAR | BENZOATE | | | example 1 |
| UVINUL T150 | ETHYLHEXYL TRIAZONE | 3 | 2 | |
| PAROSL MCX | ETHYLHEXYL METHOXYCINNAMATE | 7.5 | 2 | UV filter |
| MONTANOV 68 MB | CETEARYL ALCOHOL/CETEARYL | 6 | 2 | surfactant |
| | GLUCOSIDE | | | |
| SODIUM HYDROXIDE | SODIUM HYDROXIDE | 0.04 | 3 | neutralizer |

A comparative composition was prepared by propeller mixing ingredients in mixture 1 under 85° C. till uniform and propeller mixing ingredients in mixture 2 under 85° C. till uniform; and adding mixture 2 into mixture 1 under propeller mixing till uniform and cooled down to room temperature. The mixture 3 was added into the comparative composition by low speed homogenization at 2000-3000 rpm till uniform and cooled down to room temperature so as to provide a comparative cosmetic composition (sunscreen emulsion).

### Example 3 Stability Tests

Stability tests were taken on Example 1 and 2 over 1 month. The liquid crystal structure was checked by a polarizing microscope. The microscope used is Leica DM2500. As can be seen from table below, the liquid crystal structure for cosmetic composition of Example 1 still exists after the stability tests under RT, 50° C. and 5 cycles of freeze-thaw (−20° C. to RT to −20°° C.). The equipment used for stability test under RT and 50° C. is Binder incubator BF240 and the equipment for freeze-thaw test is Haier refrigerator DW-40L278. The liquid crystal structure for cosmetic composition of Example 2 disappeared gradually during the stability test.

This indicates stronger liquid crystal structure is obtained in present application compared with traditional liquid crystal technology.

Example 1

| stability conditions | initial | 1 week | 2 weeks | 3 weeks | 4 weeks |
|---|---|---|---|---|---|
| RT | ◯ | ◯ | ◯ | ◯ | ◯ |
| 50° C. | ◯ | ◯ | ◯ | ◯ | ◯ |
| freeze-thaw | cycle 1 | cycle 2 | cycle 3 | cycle 4 | cycle 5 |
| 1 cycle: −20° C. to | ◯ | ◯ | ◯ | ◯ | ◯ |
| RT to −20° C. | | | | | |

Comparative Example 2

| stability conditions | initial | 1 week | 2weeks | 3weeks | 4weeks |
|---|---|---|---|---|---|
| RT | ◯ | ◯ | ◯ | X | X |
| 50° C. | ◯ | X | X | X | X |
| freeze-thaw | cycle 1 | cycle 2 | cycle 3 | cycle 4 | cycle 5 |
| 1 cycle: −20° C. to | ◯ | X | X | X | X |
| RT to −20° C. | | | | | |

◯: with LC structure;
X: without LC structure
◯: with LC structure;
X: without LC structure

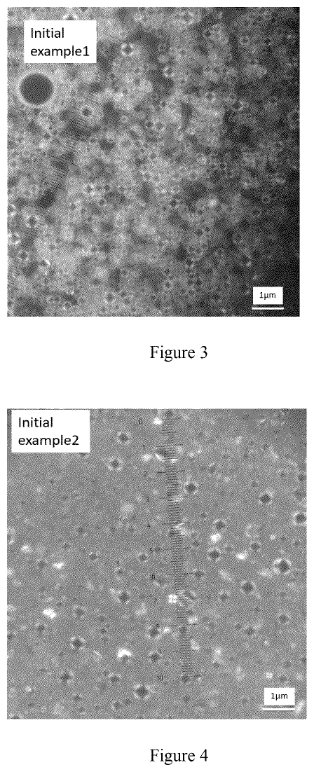

As shown in FIGS. 3 and 4, the cosmetic composition of Example 1 shows more liquid crystal particles (FIG. 3), while much fewer liquid crystal particles are observed in the comparative cosmetic composition of Example 2 by traditional liquid crystal technology (FIG. 4). The thick and sufficient Maltese crosses in FIG. 3 indicate a stronger liquid crystal structure.

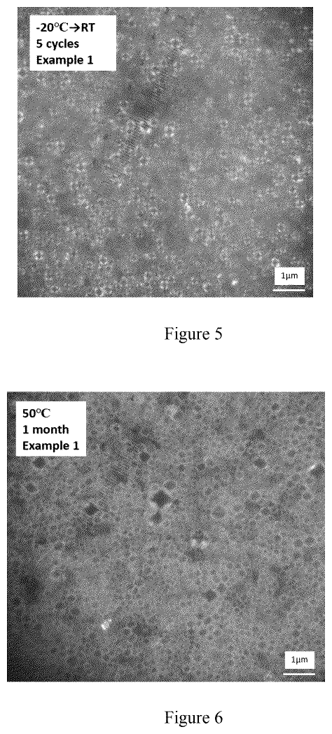

After the extreme stability tests (50° C.; freeze-thaw), liquid crystal particles in the cosmetic composition of Example 1 can still be observed as shown in FIGS. 5 and 6, while no liquid crystal particles are present anymore in comparative cosmetic composition of Example 2.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. An encapsulation composition comprising liquid crystal particles, wherein each of the liquid crystal particles comprises a core and a liquid crystal shell, wherein the core contains an active ingredient and the liquid crystal shell contains a fatty alcohol, a first surfactant, and an optional lipid.

2. The encapsulation composition of claim 1, wherein the first surfactant is a gemini surfactant, preferably a gemini amphiphilic surfactant, more preferably sodium dilauramidoglutamide lysine (DLGL), and/or the first surfactant ranges from 0.1-20%, preferably 0.1-10%, more preferably 0.1-5% by weight of the encapsulation composition.

3. The encapsulation composition of claim 1, wherein the fatty alcohol is selected from the group consisting of myristyl alcohol, cetyl alcohol, cetyl/stearyl alcohol, stearyl alcohol, arachyl alcohol, behenyl alcohol, and mixtures thereof, and/or the fatty alcohol ranges from 0.1-50%, preferably 1-20%, more preferably 1-10% by weight of the encapsulation composition.

4. The encapsulation composition of claim 1, wherein the active ingredient is lipophilic and the active ingredient ranges from 1-70%, preferably 5-65% by weight, more preferably 5-60% by weight of the encapsulation composition; preferably the active ingredient is selected from the group consisting of lipophilic ultraviolet (UV) filters, retinol and its derivatives, and mixtures thereof.

5. The encapsulation composition of claim 1, wherein the active ingredient is a lipophilic ultraviolet (UV) filter and the lipophilic ultraviolet (UV) filter ranges from 1-70%, preferably 5-65% by weight, preferably 5-60% by weight of the encapsulation composition; preferably the lipophilic ultraviolet (UV) filter is selected from the group consisting of ethylhexyl triazone, diethylamino hydroxybenzoyl hexyl benzoate, bis-ethylhexyloxyphenol methoxyphenyl triazine, and mixtures thereof.

6. The encapsulation composition of claim 1, wherein the active ingredient is selected from the group consisting of retinol and its derivatives and the retinol and its derivatives ranges from 1-70%, preferably 5-60% by weight of the encapsulation composition.

7. The encapsulation composition of claim 1, wherein the encapsulation composition is formed by mixing a first mixture comprising a polyol, the first surfactant and water and a second mixture comprising the active ingredient, the fatty alcohol and the optional lipid.

8. The encapsulation composition of claim 7, wherein the polyol is a polyol having two or more hydroxyls and 3-12, preferably 3-6 carbon atoms in the molecule; preferably the polyol is selected from the group consisting of glycerin, 1,2-propanediol, 1,3-propanediol, 1,4-butanediol, 1,3-butylene glycol, dipropylene glycol, 1,2-pentandiol, 2-methyl-pentan-2,4-diol, 1,2-hexanediol, sorbitol, 1,2-octanediol, and mixtures thereof; preferably the polyol is glycerin, sorbitol and mixtures thereof; and/or the polyol ranges from 10-80%, preferably 20-60% by weight of the encapsulation composition.

9. The encapsulation composition of claim 1, wherein the optional lipid is selected from the group consisting of triglycerides, hydrocarbons, ester, fatty acids, waxes, sphingolipids, phospholipids, cholesterols, and mixtures thereof, and/or the lipid ranges from 0-70% by weight of the encapsulation composition.

10. The encapsulation composition of claim 7, wherein the ratio by weight of the second mixture to the first mixture ranges from 0.1-10, preferably 0.2-5.

11. The encapsulation composition of claim 7, wherein the second mixture is in the form of a solid, a semi-solid, a paste, or a liquid paste at 25 degrees Celsius.

12. The encapsulation composition of claim 7, wherein the polyol comprises glycerin; the first surfactant comprises sodium dilauramidoglutamide lysine; the active ingredient comprises diethylaminohydroxybenzoyl hexyl benzoate and/or ethylhexyl triazone; and the fatty alcohol comprises behenyl alcohol.

13. The encapsulation composition of claim 1, wherein the liquid crystal shell contains the optional lipid and the active ingredient comprises retinol and/or its derivatives.

14. A cosmetic or pharmaceutical composition comprising the encapsulation composition of claim 1.

15. The cosmetic or pharmaceutical composition of claim 14, wherein the system further comprises a second surfactant and the particle size of the liquid crystal particles is in a range from 10 nm-300 μm, preferably 50 nm-100 μm, more preferably 200 nm-100 μm.

16. The cosmetic or pharmaceutical composition of claim 15, wherein the second surfactant is selected from the group consisting of fatty acid esters of glycerol, fatty acid esters of sorbitol, fatty acid ethoxylates, and mixtures thereof.

17. The cosmetic or pharmaceutical composition of claim 15, wherein the second surfactant ranges from 0.5-10%, preferably 1-10%, more preferably 1-5% by weight of total of the encapsulation composition, the at least one second surfactant and any other ingredients in the cosmetic or pharmaceutical composition.

18. A method for preparing the encapsulation composition of claim 1:
a) providing a first mixture comprising a polyol, a first surfactant, and water and a second mixture comprising an active ingredient, a fatty alcohol and an optional lipid;
b) optionally, mixing the first mixture and/or mixing the second mixture;
c) mixing the first mixture and the second mixture at a temperature equal to or higher than the melting point of the fatty alcohol;
d) combining the first and second mixtures; and
e) cooling the resulting mixture of step d) to room temperature to provide the encapsulation composition.

19-20. (canceled)
